(19) United States
(12) Patent Application Publication
(10) Pub. No.: US 2009/0119022 A1
(43) Pub. Date: May 7, 2009

(54) Emericella Nidulans Genome Sequence On Computer Readable Medium and Uses Thereof

(76) Inventors: William E. Timberlake, Bolton, MA (US); Yongwei Cao, Lexington, MA (US); Gregory J. Hinkle, Plymouth, MA (US); James D. McIninch, Burlington, MA (US); Jaehyuk Yu, North Andover, MA (US); Azita Ghodssi, Winchester, MA (US)

Correspondence Address: ARNOLD & PORTER LLP, 555 TWELFTH STREET, N.W., ATTN: IP DOCKETING, WASHINGTON, DC, 20004, US

(21) Appl. No.: 12/336,504

(22) Filed: Dec. 16, 2008

Related U.S. Application Data
(63) Continuation-in-part of application No. 09/404,520, filed on Sep. 23, 1999.
(60) Provisional application No. 60/101,665, filed on Sep. 25, 1998.
(60) Provisional application No. 60/101,666, filed on Sep. 25, 1998.
(60) Provisional application No. 60/102,358, filed on Sep. 29, 1998.
(60) Provisional application No. 60/113,361, filed on Dec. 21, 1998.
(60) Provisional application No. 60/126,265, filed on Mar. 25, 1999.
(60) Provisional application No. 60/130,189, filed on Apr. 20, 1999.
(60) Provisional application No. 60/130,190, filed on Apr. 20, 1999.
(60) Provisional application No. 60/132,861, filed on May 7, 1999.
(60) Provisional application No. 60/138,103, filed on Jun. 4, 1999.
(60) Provisional application No. 60/149,882, filed on Aug. 19, 1999.

(51) Int. Cl. G01N 33/48

(52) U.S. Cl. 702/20

(57) ABSTRACT

The present invention relates to nucleic acid sequences from the filamentous fungus, Emericella nidulans (Aspergillus nidulans) and, in particular, to genomic DNA sequences. The invention encompasses nucleic acid molecules present in non-coding regions as well as nucleic acid molecules that encode proteins and fragments of proteins. In addition, proteins and fragments of proteins so encoded and antibodies capable of binding the proteins are encompassed by the present invention. The invention also encompasses oligonucleotides including primers, e.g. useful for amplifying nucleic acid molecules, and collections of nucleic acid molecules and oligonucleotides, e.g. in microarrays. The invention also provides constructs and transgenic cells and organisms comprising nucleic acid molecules of the invention. The invention also relates to methods of using the disclosed nucleic acid molecules, oligonucleotides, proteins, fragments of proteins, and antibodies, for example, for gene identification and analysis, and preparation of constructs and transgenic cells and organisms.

This application claims priority under 35 U.S.C §119(e) of U.S. Provisional Applications Nos. 60/101,665; 60/101,666; 60/102,358; 60/113,361; 60/126,265; 60/130,189; 60/130,190; 60/132,861; 60/138,103; and 60/149,882, the disclosures of which provisional applications are incorporated herein by reference in their entirety.

### FIELD OF THE INVENTION

Included in the disclosure are nucleic acid molecules representing the genome of the filamentous fungus, Emericella nidulans (previously and still sometimes called Aspergillus nidulans) and, in particular, to nucleic acid molecules having nucleic acid sequences corresponding to genes, promoters, other regulatory elements, and introns found in the E. nidulans genome, a specific set of genes of E. nidulans and a set of primers based on the E. nidulans genes. Also disclosed are homologous nucleic acid molecules, complementary nucleic acid molecules, polypeptides expressed by such genes, constructs comprising such promoters, regulatory elements and/or genes, transformed cells and organisms comprising such genes and/or promoters and regulatory elements, primers useful for replicating parts of such genes and nucleic acid molecules, computer readable media comprising sets of such nucleic acid sequences, polypeptides and primers, collections of nucleic acid molecules and methods of using such molecules and sequences including the use of collections of nucleic acid molecules in genetic research and clinical analysis, e.g. for gene expression.

### BACKGROUND OF THE INVENTION

Filamentous fungi have a complex multicellular organization involving production of highly specialized cell types as part of their normal asexual and sexual lifecycles. Fungi as experimental systems are good models for plant and animal cell functions because of their evolutionary relatedness. E. nidulans is a model eukaryotic organism and has been used extensively to address fundamental questions of biology. E. nidulans is a more complex organism than yeast and has many genes which have a similar function to genes found implants and animals. This filamentous fungus has been employed in investigations into a variety of genetic phenomena including the mechanisms regulating carbon and nitrogen metabolism, cell cycle, cytoskeletal functions, and development. A set of nucleic acid molecules representing substantially most of the genes in the E. nidulans genome is useful in transcription profiling work to find, identify and characterize counterpart genes in other species, particularly microbial and plant species. For instance, it is possible to identify unknown plant gene function by studying a similar (homologous) gene in a microbe in which genetic modification can more easily be done. That is, if unknown genes are disrupted or overexpressed, transcription profiling can be carried out to understand effects of the genetic modification.

Moreover, chemical/drug discovery can be practiced using such transcription profiling with nucleic acids molecules of the E. nidulans genome. And, because many human or plant pathogens are filamentous fungi and E. nidulans is a model organism for filamentous fungi, transcription profiling with genome-wide expression of the E. nidulans genome is an efficient way to understand the action of such pathogens and their secondary metabolites, e.g. mycotoxins which can be deleterious to food and feed supplies. In addition environmental stress studies of the E. nidulans genome will provide insight into related mechanisms in plants, e.g. yield, stability, thermal resistance, water/drought tolerance, etc.

Nucleic acid molecules comprising the E. nidulans genome disclosed herein were identified and isolated from a sample of filamentous fungus identified as Aspergillus nidulans, FGSC Number A4, obtained from the Fungal Genetics Stock Center (FGSC) at the University of Kansas Medical Center, Kansas City, Kans. It has been determined that this fungus is more properly named Emericella, nidulans. As used herein the terms Emericella nidulans, E. nidulans, Aspergillus nidulans and A. nidulans refer to the filamentous fungus previously and still sometimes called Aspergillus nidulans.

Nucleic acid sequences of a species, e.g. the E. nidulans, can be generated by random shotgun sequencing of cloned genomic DNA and assembled into longer lengths of contiguous sequence (contigs). The final data set from an assembly process comprises a collection of sequences, which includes the contigs resulting from linking of two or more overlapping sequences as well as singleton nucleic acid sequences, i.e. trace sequences which are not incorporated into contigs. Such sequences can be screened for genes, e.g. full length or substantially full length or partial length genes. Screening methods include homology searches against databases of known genes and predictive methods using algorithms which infer the presence and extent of a gene.

The nucleic acid sequences disclosed herein are believed to represent substantially all, or at least a major part, of the genes in the E. nidulans genome. Genome sequence information from E. nidulans permits identification of genetic sequences from other organisms, including plants, mammals such as humans, bacteria, other filamentous fungi and non-filamentous fungi such as a yeast, e.g. by comparison of such sequences with E. nidulans sequences. The availability of a substantially complete set genes or partial genes of the E. nidulans genome permits the definition of primers for fabricating representative nucleic acid molecules of the genome which can be used on microarrays facilitating transcription profile studies. In addition the identification of the E. nidulans genome permits the fabrication of a wide variety of DNA constructs useful for imparting unique genetic properties into transgenic organisms. These and other advantages attendant with the various aspects of this invention will be apparent from the following description of the invention and its various embodiments.

### SUMMARY OF THE INVENTION

The present invention contemplates and provides a substantial part of the genome of the filamentous fungus Emericella nidulans. One aspect of the invention is a set of more than 16,000 contig and singleton sequences comprising coding sequence as well as promoters, other regulatory elements and introns represented by SEQ ID NO: 1 through SEQ ID NO: 16206. Contigs in SEQ ID NO: 1 through SEQ ID NO: 16206 are recognized as those sequences whose designations begin with ANI61C or ANI50C. Singleton sequences are recognized as those having designations which begin with ANI61S or ANI50S. Thus, a subset of the nucleic acid molecules of this invention comprises promoters and/or other regulatory elements of the E. nidulans genome as present in SEQ ID NO: 1 through SEQ ID NO: 16206 or complements thereof.

Another aspect of this invention comprises a set of about 12,000 genes or partial genes of the E. nidulans genome including genes represented by SEQ ID NO: 16207 through SEQ ID NO: 27905 and a small set of previously reported genes represented by SEQ ID NO: 27906 through SEQ ID NO: 28165. As used herein, a substantially complete set of genes for an organism is referred to as a unigene set. Thus, as used herein reference is made to specific genes comprising the unigene set of E. nidulas as “ENUxxxxx” where ENU is an acronym for Emericella nidulans unigene and xxxxx represents a number. Thus, ENU0001 to ENU27905 are used to designate the genes of E. nidulans identified herein; and, ENU27906 to ENU28165 are used to designate the previously reported genes of E. nidulans. Moreover, the term “ENU” by itself is also used herein to mean any of the nucleic acid molecules comprising genes or partial genes of the unigene set for E. nidulans. More particularly the term “ENU of this invention” as used herein means a nucleic acid molecule representing a gene or partial gene of E. nidulans disclosed herein selected from the group consisting of ENU00001 to ENU27905.

The present invention also contemplates and provides substantially purified nucleic acid molecules comprising the ENUs and other nucleic acid molecules of this invention as well as molecules which are complementary to, and capable of specifically hybridizing to, the ENU or its complement.

The present invention also contemplates and provides substantially purified nucleic acids molecules which are homologous to the nucleic acid molecules of this invention including, for example, those which are homologous to the ENUs of this invention, e.g. a plurality of related sets of homologous nucleic acid molecules in other species which are homologous to the ENUs.

The present invention also contemplates and provides substantially purified protein, or polypeptide fragments thereof, which are encoded by cDNA associated with the ENUs of the present invention.

The present invention also contemplates and provides constructs comprising promoters, regulatory elements and/or the ENUs which are useful in making transgenic cells or organisms. In particular this invention also provides transformed cell or organism having a nucleic acid molecule which comprises: (a) a promoter region which functions in the cell to cause the production of a mRNA molecule; which is linked to (b) a structural nucleic acid molecule, which is linked to (c) a 3′ non-translated sequence that functions in the cell to cause termination of transcription and addition of polyadenylated ribonucleotides to a 3′ end of the mRNA molecule, where components (a) and/or (b) are selected from E. nidulans nucleic acid sequences provided herein and more preferably selected E. nidulans nucleic acid sequences from the group consisting of ENU00001 to ENU27905.

Still another aspect of this invention is a set (and subsets thereof) of about 24,000 primers for the ENUs of this invention, including a specific subset of about 16,000 primers represented by SEQ ID NO: 28166 through SEQ ID NO: 44345 which can be used to generate and isolate nucleic acid molecules representative ENUs of this invention and homologs thereof in other non-E. Nidulans species. The nucleic acids molecules of this invention including primers represent a useful tool in genetic research not only for the species E. nidulans, but also for other fungal species, other microorganisms and life forms with more differentiated cell structure such as plants and animals. The present invention also contemplates and provides primer pairs for replicating or identifying parts of the ENUs.

The present invention also contemplates and provides computer readable media having recorded thereon one or more of the nucleotide sequences provided by this invention and methods for using such media, e.g. in searching to identify genes associated with nucleic acid sequences.

The present invention also contemplates and provides collections of nucleic acid molecules, including oligonucleotides, representing the E. nidulans genome including collections on solid substrates, e.g. substrates having attached thereto in array form nucleic acid molecules or oligonucleotides representing genes of the E. nidulans genome. The invention also contemplates and provides methods of using such collections and arrays, e.g. in transcription profiling analysis. The present invention also contemplates and provides methods for using the nucleic acid molecules of this invention, e.g. for identifying genetic material and/or determining gene expression by hybridizing expressed and labeled nucleic acid molecules or fragments thereof to arrayed collections of the nucleic acid molecules of this invention.

The present invention also contemplates and provides oligonucleotides which are identical or complementary to a sequence of similar length for an ENU. Such oligonucleotides are useful, for example, for hybridzing to and identifying nucleic acid molecules which are homologous and/or complemetary to the ENUs of the present invention.

### DETAILED DESCRIPTION OF THE INVENTION

As used herein, a nucleic acid molecule and/or polypeptide molecule, be it a naturally occurring molecule or otherwise, may be “substantially purified”, if the molecule is separated from substantially all other molecules normally associated with it in its native state. More preferably a substantially purified molecule is the predominant species present in a preparation. A substantially purified molecule may be greater than 60% free, preferably 75% free, more preferably 90% free, and most preferably 95% free from the other molecules (exclusive of solvent) present in the natural mixture. The term “substantially purified” is not intended to encompass molecules present in their native state.

The ENUs of this invention and other nucleic acid molecules and/or polypeptide molecules of the present invention will preferably be “biologically active” with respect to either a structural attribute, such as the capacity of a nucleic acid to hybridize to another nucleic acid molecule, or the ability of a protein to be bound by an antibody (or to compete with another molecule for such binding). Alternatively, such an attribute may be catalytic, and thus involve the capacity of the agent to mediate a chemical reaction or response.

As used herein the term “polypeptide” means a protein or fragment thereof expressed by a nucleic acid molecule in a cell.

The ENUs of this invention and other nucleic acid molecules of the present invention may also be recombinant. As used herein, the term recombinant means any molecule (e.g. DNA, peptide etc.), that is, or results, however indirect, from human manipulation of a nucleic acid molecule.

It is understood that the nucleic acid molecules of the present invention may be labeled with reagents that facilitate detection of the agent, e.g. fluorescent labels as disclosed in U.S. Pat. No. 4,653,417, chemical labels as disclosed in U.S. Pat. Nos. 4,582,789 and 4,563,417 and modified bases as disclosed in U.S. Pat. No. 4,605,735, all of which are incorporated herein by reference in their entirety.

The term “oligonucleotide” as used herein refers to short nucleic acid molecules useful, e.g. for hybridizing probes, nucleotide array elements or amplification primers. Oligonucletide molecules are comprised of two or more nucleotides, i.e. deoxyribonucleotides or ribonucleotides, preferably more than five and up to 30 or more. The exact size will depend on many factors, which in turn depend on the ultimate function or use of the oligonucleotide. Oligonucleotides can comprise ligated natural nucleic molecules acids or synthesized nucleic acid molecules and comprise between 5 to 150 nucleotides or between about 15 and about 100 nucleotides, or preferably up to 100 nucleotides, and even more preferably between 15 to 30 nucleotides or most preferably between 18-25 nucleotides, identical or complementary to a sequence of similar length for an ENU.

This invention provides oligonucleotides specific for ENU sequences. Such oligonucleotides may be nucleic acid elements for use on solid arrays (e.g. synthesized or spotted) or primers for amplification of ENUs of this invention. Such primers for use in polymerase chain reaction (PCR) primers are preferably designed with the goal of amplifying nucleic acids from either the 3′ or the 5′ end of an ENU or a fragment of an ENU, e.g. about 500 to 800 bp of nucleic acids from the at the 3′ end of such a nucleic acid molecule.

The term “primer” as used herein refers to a nucleic acid molecule, preferably an oligonucleotide whether derived from a naturally occurring molecule, such as one isolated from a restriction digest or one produced synthetically, which is capable of acting as a point of initiation of synthesis when placed under conditions in which synthesis of a primer extension product which is complementary to a nucleic acid strand is induced, i.e., in the presence of nucleotides and an agent for polymerization such as DNA polymerase and at a suitable temperature and pH. The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the agent for polymerization. The exact lengths of the primers will depend on many factors, including temperature and source of primer. For example, depending on the complexity of the target sequence, the oligonucleotide primer typically contains at least 15, more preferably 18 nucleotides, which are identical or complementary to the template and optionally a tail of variable length which need not match the template. The length of the tail should not be so long that it interferes with the recognition of the template. Short primer molecules generally require cooler temperatures to form sufficiently stable hybrid complexes with the template.

The primers herein are selected to be “substantially” complementary to the different strands of each specific sequence to be amplified. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to be amplified to hybridize therewith and thereby form a template for synthesis of the extension product of the other primer. Computer generated searches using programs such as Primer3 (www-genome.wi.mit.edu/cgi-bin/primer/primer3.cgi), STSPipeline (www-genome.wi.mit.edu/cgi-bin/www-STS_Pipeline), or GeneUp (Pesole et al., BioTechniques 25:112-123 (1998)), for example, can be used to identify potential PCR primers. Exemplary primers include primers that are 18 to 50 bases long, where at least between 18 to 25 bases are identical or complementary to at least 18 to 25 bases segment of the template sequence. Preferred template sequences for such primers are selected from a fragment of any one of SEQ ID NO: 16207 through SEQ ID NO: 28905 or complements thereof.

This invention also contemplates and provides primer pairs for amplification of nucleic acid molecules representing the ENUs. As used herein “primer pair” means a set of two oligonucleotide primers based on two separated sequence segments of a target nucleic acid sequence. One primer of the pair is a “forward primer” or “5′ primer” having a sequence which is identical to the more 5′ of the separated sequence segments. The other primer of the pair is a “reverse primer” or “3′ primer” having a sequence which is complementary to the more 3′ of the separated sequence segments. A primer pair allows for amplification of the nucleic acid sequence between and including the separated sequence segments. Optionally, each primer pair can comprise additional sequences, e.g. universal primer sequences or restriction endonuclease sites, at the 5′ end of each primer, e.g. to facilitate cloning, DNA sequencing, or reamplification of the target nucleic acid sequence.

Nucleic acid molecules of the present invention include those having a nucleic acid sequence selected from the group consisting of SEQ ID NO: 1 though SEQ ID NO: 44,435 and complements thereof and fragments of either. Preferred nucleic acid molecules include those having a nucleic acid sequence selected from the following groups: SEQ ID NO: 16207 through SEQ ID NO: 27905 or complements thereof; SEQ ID NO: 16207 through SEQ ID NO: 26804 or complements thereof; SEQ ID NO: 26000 through SEQ ID NO: 26804 or complements thereof; SEQ ID NO: 16207 through SEQ ID NO: 25999 or complements thereof; SEQ ID NO: 24035 through SEQ ID NO: 25999 or complements thereof; SEQ ID NO: 16207 through SEQ ID NO: 24034 or complements thereof; SEQ ID NO: 22710 through SEQ ID NO: 24034 or complements thereof; SEQ ID NO: 16207 through SEQ ID NO: 22709 or complements thereof; SEQ ID NO: 17681 through SEQ ID NO: 22709 or complements thereof; SEQ ID NO: 16207 through SEQ ID NO: 17680 or complements thereof; SEQ ID NO: 17618 through SEQ ID NO: 17680 or complements thereof; SEQ ID NO: 16207 through SEQ ID NO: 17617 or complements thereof; SEQ ID NO: 17295 through SEQ ID NO: 17617 or complements thereof; SEQ ID NO: 16207 through SEQ ID NO: 17294 or complements thereof. Other preferred nucleic acid molecules include any of the above groups but where such groups also include fragments of such sequences.

Nucleic acid molecules or fragments thereof are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure along a sufficient portion of the molecule to allow for stable binding under laboratory hybridizing conditions. A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., Molecular Cloning, A Laboratory Manual, 2nd Ed., Cold Spring Harbor Press, Cold Spring Harbor, N.Y. (1989), and by Haymes et al., Nucleic Acid Hybridization, A Practical Approach, IRL Press, Washington, D.C. (1985), the entirety of both of which are herein incorporated by reference. Departures from complete complementarity are therefore permissible, as long as such departures do not completely preclude the capacity of the molecules to form a double-stranded structure. Thus, in order for a nucleic acid molecule to serve as a primer or probe it need only be sufficiently complementary in sequence to be able to form a stable double-stranded structure under the particular solvent and salt concentrations employed.

Appropriate stringency conditions which promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

Preferred embodiments of the nucleic acid of this invention will specifically hybridize to one or more of the ENUs of this invention or complements thereof under low stringency conditions, for example at about 2.0×SSC and about 50° C. In a particularly preferred embodiment, a nucleic acid of the present invention will include those nucleic acid molecules that specifically hybridize to one or more of the ENUs of this invention or complements thereof under moderate stringency conditions. In an especially preferred embodiment, a nucleic acid of the present invention will include those nucleic acid molecules that specifically hybridize to one or more of the ENUs of this invention or complements thereof under high stringency conditions.

In another aspect of the present invention, one or more of the nucleic acid molecules of the present invention share between 100% and 90% sequence identity with one or more of the ENUs of this invention or complements thereof. In a further aspect of the present invention, one or more of the nucleic acid molecules of the present invention share between 100% and 95% sequence identity with one or more of the ENUs of this invention or complements thereof. In a more preferred aspect of the present invention, one or more of the nucleic acid molecules of the present invention share between 100% and 98% sequence identity with one or more of the ENUs of this invention or complements thereof. In an even more preferred aspect of the present invention, one or more of the nucleic acid molecules of the present invention share between 100% and 99% sequence identity with one or more of the ENUs of this invention or complements thereof.

The present invention also encompasses the use of nucleic acids of the present invention in recombinant constructs. Using methods known to those of ordinary skill in the art, an ENU sequence and/or a promoter sequence of the invention can be inserted into constructs which can be introduced into a host cell of choice for expression of the encoded protein if an ENU is used or for use of an E. nidulans promoter to direct expression of a heterologous protein. Potential host cells include both prokaryotic and eukaryotic cells. A host cell may be unicellular or found in a multicellar differentiated or undifferentiated organism depending upon the intended use. It is understood that useful exogenous genetic material may be introduced into any non-fungal cell or organism such as a plant cell, plant, mammalian cell, mammal, fish cell, fish, bird cell, bird or bacterial cell.

Depending upon the host, the regulatory regions for expression of ENU sequences will vary, including regions from viral, plasmid or chromosomal genes, or the like. For expression in prokaryotic or eukaryotic microorganisms, particularly unicellular hosts, a wide variety of constitutive or regulatable promoters may be employed. Among transcriptional initiation regions which have been described are regions from bacterial and yeast hosts, such as E. coli, B. subtilis, Sacchromyces cerevisiae, including genes such as beta-galactosidase, T7 polymerase and tryptophan E.

Furthermore, for use in transformation of E. nidulans, constructs may include those in which an ENU sequence or portion thereof of the present invention is positioned with respect to a promoter sequence such that production of antisense mRNA complementary to native mRNA molecules is provided. In this manner, expression of the native gene may be decreased. Such methods may find use for modification of particular functions of the targeted host, and/or for discovering the function of a protein naturally expressed in E. nidulans.

### Complements and Homologs of ENUs

Another embodiment of the present invention comprises a nucleic acid molecule which is a homolog of an ENU of this invention which encodes a polypeptide also found in a plant, animal or bacterial organism. Yet another embodiment comprises a nucleic acid molecule which encodes a polypeptide which is homologous to a polypeptide encoded by an ENU of this invention where the percent identity between the polypeptides is between about 25% and about 40%, more preferably of between about 40 and about 70%, even more preferably of between about 70% and about 90%, and even more preferably between about 90% and 99% and most preferably 100%.

Genomic sequences can be screened for the presence of protein homologs utilizing one or a number of different search algorithms that have been developed, one example of which are the suite of programs referred to as BLAST programs. In addition, unidentified reading frames may be screened for by gene prediction software such as GenScan available for downloading from the Stanford University web site. The degeneracy of the genetic code allows different nucleic acid sequences to code for the same protein or peptide, e.g. see U.S. Pat. No. 4,757,006, the entirety of which is herein incorporated by reference. As used herein a nucleic acid molecule is degenerate of another nucleic acid molecule when the nucleic acid molecules encode for the same amino acid sequences but comprise different nucleotide sequences. An aspect of the present invention is that the nucleic acid molecules of the present invention include nucleic acid molecules that are degenerate from the ENUs of this invention.

A further aspect of the present invention comprises one or more nucleic acid molecules which differ in nucleic acid sequence from those of an ENU of this invention due to the degeneracy in the genetic code in that they encode the same protein but differ in nucleic acid sequence or a protein having one or more conservative amino acid residue. Codons capable of coding for such conservative substitutions are known in the art. For instance, serine is a conservative substitute of alanine and threonine is a conservative substitute for serine.

Regulatory Elements

One class of agents of the present invention includes nucleic acid molecules having promoter regions or partial promoter regions or other regulatory elements, particularly those found in SEQ ID NO: 1 through SEQ ID NO: 16144 and located upstream of the trinucleotide ATG sequence at the start site of a protein coding region. As used herein, a promoter region is a region of a nucleic acid molecule that is capable, when located in cis to a nucleic acid sequence that encodes for a protein or peptide to function in a way that directs expression of one or more mRNA molecules that encodes for the protein or peptide. Promoters of the present invention can comprise nucleic acids in the range from about 300 bp to at least 1000 bp or more, say about 2000 bp or even higher say about 5000 bp and up to about 10 kb upstream of the trinucleotide ATG sequence at the start site of a protein coding region. While in many circumstances a 300 bp promoter may be sufficient for expression, additional sequences may act to further regulate expression, for example, in response to biochemical, developmental or environmental signals. In a preferred embodiment of the present invention, the promoter is upstream of a nucleic acid sequence that encodes an E. nidulans protein homolog or fragment thereof or preferably upstream of an ENU of this invention. It is also preferred that the promoters of the present invention contain a CAAT and a TATA cis element. Moreover, the promoters of the present invention can include one or more cis elements in addition to a CAAT and a TATA box. For the most part, the promoters of the present invention will be located in contig sequences which generally represent longer nucleic acids than do singleton sequences of the present invention. Contigs in SEQ ID NO:1 through SEQ ID NO:16144 are recognized as those sequences whose designations begin with ANI61C or ANI50C, as opposed to singletons whose designations begin with ANI61S or ANI50S. Where an ENU is specified as being located on two different contigs, the promoter region will be located on the contig representing the 5′ region of the gene encoding sequence.

By “regulatory element” it is intended a series of nucleotides that determines if, when, and at what level a particular gene is expressed. The regulatory DNA sequences specifically interact with regulatory or other proteins. Many regulatory elements act in cis (“cis elements”) and are believed to affect DNA topology, producing local conformations that selectively allow or restrict access of RNA polymerase to the DNA template or that facilitate selective opening of the double helix at the site of transcriptional initiation. Cis elements occur within, but are not limited to promoters, and promoter modulating sequences (inducible elements). Cis elements can be identified using known cis elements as a target sequence or target motif in the BLAST programs of the present invention. Promoters of the present invention include homologs of cis elements known to effect gene regulation that show homology with the nucleic acid molecules of the present invention.

Polypeptides

Other aspects of this invention comprises one or more of the polypeptides, including proteins or peptide molecules, encoded by the coding region of an ENU of this invention or fragments thereof or homologs thereof. Protein and peptide molecules can be identified using known protein or peptide molecules as a target sequence or target motif in the BLAST programs of the present invention. In a preferred embodiment the protein or fragment molecules of the present invention are derived from E. nidulans.

As used herein, the term “protein molecule” or “peptide molecule” includes any molecule that comprises five or more amino acids. It is well known in the art that proteins or peptides may undergo modification, including post-translational modifications, such as, but not limited to, disulfide bond formation, glycosylation, phosphorylation, or oligomerization. Thus, as used herein, the term “protein molecule” or “peptide molecule” includes any protein molecule that is modified by any biological or non-biological process. The terms “amino acid” and “amino acids” refer to all naturally occurring L-amino acids. This definition is meant to include norleucine, ornithine, homocysteine, and homoserine.

One or more of the protein or peptide molecules may be produced via chemical synthesis, or more preferably, by expression in a suitable bacterial or eukaryotic host. Suitable methods for expression are described by Sambrook et al., Molecular Cloning, A Laboratory Manual, 2nd Edition, Cold Spring Harbor Press, Cold Spring Harbor, N.Y. (1989), or similar texts.

A “protein fragment” comprises a subset of the amino acid sequence of that protein. A protein fragment which comprises one or more additional peptide regions not derived from a base protein is a “fusion” protein. Such molecules may be derivatized to contain carbohydrate or other groups (such as keyhole limpet hemocyanin, etc.). Fusion protein or peptide molecules of the present invention are preferably produced via recombinant means.

Another class of agents comprises protein or peptide molecules encoded by the coding region of an ENU of this invention or complements thereof or, fragments or fusions thereof in which conservative, non-essential, or not relevant, amino acid residues have been added, replaced, or deleted. An example of such a homolog is the homolog protein of a non-E. nidulans filamentous fungus. Such a homolog can be obtained by any of a variety of methods. For example, as indicated above, one or more of the disclosed sequences for primers of this invention can be used to define a pair of primers that may be used to isolate the homolog-encoding nucleic acid molecules from any desired species. Such molecules can be expressed to yield homologs by recombinant means.

Antibodies

One aspect of the present invention concerns antibodies, single-chain antigen binding molecules, or other proteins that specifically bind to one or more of the protein or peptide molecules of the present invention and their homologs, fusions or fragments. Such antibodies may be used to quantitatively or qualitatively detect the protein or peptide molecules of the present invention. As used herein, an antibody or peptide is said to “specifically bind” to a protein or peptide molecule of the present invention if such binding is not competitively inhibited by the presence of non-related molecules. In a preferred embodiment the antibodies of the present invention bind to proteins of the present invention, in a more preferred embodiment of the antibodies of the present invention bind to proteins derived from E. nidulans.

Nucleic acid molecules that encode all or part of the protein of the present invention can be expressed, via recombinant means, to yield protein or peptides that can in turn be used to elicit antibodies that are capable of binding the expressed protein or peptide. Such antibodies may be used in immunoassays for that protein. Such protein-encoding molecules, or their fragments may be a “fusion” molecule (i.e., a part of a larger nucleic acid molecule) such that, upon expression, a fusion protein is produced. It is understood that any of the nucleic acid molecules of the present invention may be expressed, via recombinant means, to yield proteins or peptides encoded by these nucleic acid molecules.

The antibodies that specifically bind proteins and protein fragments of the present invention may be polyclonal or monoclonal. It is understood that practitioners are familiar with the standard resource materials which describe specific conditions and procedures for the construction, manipulation and isolation of antibodies (see, for example, Harlow and Lane, Antibodies: A Laboratory Manual, Cold Spring Harbor Press, Cold Spring Harbor, N.Y. (1988), the entirety of which is herein incorporated by reference).

It is understood that any of the antibodies of the present invention can be substantially purified and/or be biologically active and/or recombinant.

Fungal Constructs and Fungal Transformants

The present invention also relates to a fungal recombinant vector, e.g. comprising exogenous genetic material. In a preferred embodiment the exogenous genetic material includes at least one nucleic acid molecule of the present invention which can preferably be (a) an ENU of this invention or fragment or homolog thereof or (b) a regulatory element, promoter or partial promoter of the present invention. In a further more preferred embodiment of the present invention exogenous genetic material includes a regulatory element, promoter or partial promoter of the present invention and a nucleic acid molecule of the present invention having a sequence within a contig selected from the group identified by SEQ ID NO: 1 through SEQ ID NO: 16206 or complements thereof or fragments of either. In a further more preferred embodiment of the present invention exogenous genetic material includes a regulatory element, promoter or partial promoter of the present invention and a nucleic acid molecule encoding an E. nidulans protein homolog or fragments thereof. It is also understood that such exogenous genetic material may be introduced into any non-fungal cell or organism such as a plant cell, plant, mammalian cell, mammal, fish cell, fish, bird cell, bird or bacterial cell.

The recombinant vector may be any vector which can be conveniently subjected to recombinant DNA procedures. The choice of a vector will typically depend on the compatibility of the vector with the host cell into which the vector is to be introduced. The vector may be a linear or a closed circular plasmid. The vector system may be a single vector or plasmid or two or more vectors or plasmids which together contain the total DNA to be introduced into the genome of the host.

The vectors of the present invention preferably contain one or more selectable markers which permit easy selection of transformed cells. A selectable marker is a gene the product of which provides, for example biocide or viral resistance, resistance to heavy metals, prototrophy to auxotrophs, and the like. The selectable marker may be selected from the group including, but not limited to, amdS (acetamidase), argB (ornithine carbamoyltransferase), bar (phosphinothricin acetyltransferase), hygB (hygromycin phosphotransferase), niaD (nitrate reductase), pyrG (orotidine-5′-phosphate decarboxylase), sC (sulfate adenyltransferase), trpC (anthranilate synthase) and gfp (green fluorescent protein). Preferred for use in an Emericella cell are the amdS and pyrG markers of Emericella nidulans or Aspergillus, oryzae and the bar marker of Streptomyces hygroscopicus. Furthermore, selection may be accomplished by co-transformation, e.g., as described in WO 91/17243, the entirety of which is herein incorporated by reference.

A nucleic acid sequence of the present invention may be operably linked to a suitable promoter sequence. A protein or fragment thereof encoding nucleic acid molecule of the present invention may also be operably linked to a suitable leader sequence. A leader sequence is a nontranslated region of a mRNA which is important for translation by the fungal host. The leader sequence is operably linked to the 5′ terminus of the nucleic acid sequence encoding the protein or fragment thereof. The leader sequence may be native to the nucleic acid sequence encoding the protein or fragment thereof or may be obtained from foreign sources. A polyadenylation sequence may also be operably linked to the 3′ terminus of the nucleic acid sequence of the present invention.

To avoid the necessity of disrupting the cell to obtain the protein or fragment thereof, and to minimize the amount of possible degradation of the expressed protein or fragment thereof within the cell, it may be preferred that expression of the protein or fragment thereof gives rise to a product secreted outside the cell, especially in the case of expression in host cells of fungus or bacteria. To this end, the protein or fragment thereof of the present invention may be linked to a signal peptide linked to the amino terminus of the protein or fragment thereof. A signal peptide is an amino acid sequence which permits the secretion of the protein or fragment thereof from the host into the culture medium.

A protein or fragment thereof encoding nucleic acid molecule of the present invention may also be linked to a propeptide coding region. A propeptide is an amino acid sequence found at the amino terminus of aproprotein or proenzyme. Cleavage of the propeptide from the proprotein yields a mature biochemically active protein. The resulting polypeptide is known as a propolypeptide or proenzyme (or a zymogen in some cases). Propolypeptides are generally inactive and can be converted to mature active polypeptides by catalytic or autocatalytic cleavage of the propeptide from the propolypeptide or proenzyme. The propeptide coding region may be native to the protein or fragment thereof or may be obtained from foreign sources.

The expressed protein or fragment thereof may be detected using methods known in the art that are specific for the particular protein or fragment. These detection methods may include the use of specific antibodies, formation of an enzyme product, or disappearance of an enzyme substrate. For example, if the protein or fragment thereof has enzymatic activity, an enzyme assay may be used. Alternatively, if polyclonal or monoclonal antibodies specific to the protein or fragment thereof are available, immunoassays may be employed using the antibodies to the protein or fragment thereof. The techniques of enzyme assay and immunoassay are well known to those skilled in the art.

The resulting protein or fragment thereof may be recovered by methods known in the arts For example, the protein or fragment thereof may be recovered from the nutrient medium by conventional procedures including, but not limited to, centrifugation, filtration, extraction, spray-drying, evaporation, or precipitation. The recovered protein or fragment thereof may then be further purified by a variety of chromatographic procedures, e.g., ion exchange chromatography, gel filtration chromatography, affinity chromatography, or the like.

Plant Constructs and Plant Transformants

ENUs or other nucleic acid molecules of this invention may be used in plant transformation or transfection. Exogenous genetic material may be transferred into a plant cell and the plant cell regenerated into a whole, fertile or sterile plant. Exogenous genetic material is any genetic material, whether naturally occurring or otherwise, from any source that is capable of being inserted into any organism. Such genetic material may be transferred into either monocotyledons and dicotyledons including but not limited to the plants, alfalfa, Arabidopsis thaliana, barley, broccoli, cabbage, citrus, cotton, garlic, oat, oilseed rape, onion, canola, flax, maize, an ornamental plant, pea, peanut, pepper, potato, rice, rye, sorghum, soybean, strawberry, sugarcane, sugarbeet, tomato, wheat, poplar, pine, fir, eucalyptus, apple, lettuce, lentils, grape, banana, tea, turf grasses, sunflower, oil palm, etc.

Exogenous genetic material may be transferred into a plant cell by the use of a DNA vector or construct designed for such a purpose. Vectors have been engineered for transformation of large DNA inserts into plant genomes. Binary bacterial artificial chromosomes have been designed to replicate in both E. coli and Agrobacterium tumefaciens and have all of the features required for transferring large inserts of DNA into plant chromosomes. BAC vectors, e.g. a pBACwich, have been developed to achieve site-directed integration of DNA into a genome.

A construct or vector may also include a plant promoter to express the protein or protein fragment of choice. A number of promoters which are active in plant cells have been described in the literature. These include the nopaline synthase (NOS) promoter, the octopine synthase (OCS) promoter, a caulimovirus promoter such as the CaMV 19S promoter and the CaMV 35S promoter, the figwort mosaic virus 35S promoter, the light-inducible promoter from the small subunit of ribulose-1,5-bis-phosphate carboxylase (ssRUBISCO), the Adh promoter, the sucrose synthase promoter, the R gene complex promoter, and the chlorophyll a/b binding protein gene promoter. For the purpose of expression in source tissues of the plant, such as the leaf, seed, root or stem, it is preferred that the promoters utilized in the present invention have relatively high expression in these specific tissues. For this purpose, one may choose from a number of promoters for genes with tissue- or cell-specific or -enhanced expression. Examples of such promoters reported in the literature include the chloroplast glutamine synthetase GS2 promoter from pea, the chloroplast fructose-1,6-biphosphatase (FBPase) promoter from wheat, the nuclear photosynthetic ST-LS1 promoter from potato, the phenylalanine ammonia-lyase (PAL) promoter and the chalcone synthase (CHS) promoter from Arabidopsis thaliana. Also reported to be active in photosynthetically active tissues are the ribulose-1,5-bisphosphate carboxylase (RbcS) promoter from eastern larch (Larix laricina), the promoter for the cab gene, cab6, from pine, the promoter for the Cab-1 gene from wheat, the promoter for the CAB-1 gene from spinach, the promoter for the cab1R gene from rice, the pyruvate, orthophosphate dikinase (PPDK) promoter from Zea mays, the promoter for the tobacco Lhcb1*2 gene, the Arabidopsis thaliana SUC2 sucrose-H⁺ symporter promoter, and the promoter for the thylacoid membrane proteins from spinach (psaD, psaF, psaE, PC, FNR, atpC, atpD, cab, rbcS). Other promoters for the chlorophyl a/b-binding proteins may also be utilized in the present invention, such as the promoters for LhcB gene and PsbP gene from white mustard (Sinapis alba). Additional promoters that may be utilized are described, for example, in U.S. Pat. Nos. 5,378,619; 5,391,725; 5,428,147; 5,447,858; 5,608,144; 5,608,144; 5,614,399; 5,633,441; 5,633,435 and 4,633,436, all of which are herein incorporated in their entirety.

Constructs or vectors may also include, with the coding region of interest, a nucleic acid sequence that acts, in whole or in part, to terminate transcription of that region. For example, such sequences have been isolated including the Tr7 3′ sequence and the nos 3′ sequence or the like. It is understood that one or more sequences of the present invention that act to terminate transcription may be used.

A vector or construct may also include other regulatory elements or selectable markers. Selectable markers may also be used to select for plants or plant cells that contain the exogenous genetic material. Examples of such include, but are not limited to, a neo gene which codes for kanamycin resistance and can be selected for using kanamycin, G418, etc.; a bar gene which codes for bialaphos resistance; a mutant EPSP synthase gene which encodes glyphosate resistance; a nitrilase gene which confers resistance to bromoxynil, a mutant acetolactate synthase gene (ALS) which confers imidazolinone or sulphonylurea resistance; and a methotrexate resistant DHFR gene.

A vector or construct may also include a screenable marker to monitor expression. Exemplary screenable markers include a β-glucuronidase or uidA gene (GUS), an R-locus gene, which encodes a product that regulates the production of anthocyanin pigments (red color) in plant tissues; a β-lactamase gene, a gene which encodes an enzyme for which various chromogenic substrates are known (e.g., PADAC, a chromogenic cephalosporin); a luciferase gene, a xylE gene which encodes a catechol dioxygenase that can convert chromogenic catechols; an α-amylase gene, a tyrosinase gene which encodes an enzyme capable of oxidizing tyrosine to DOPA and dopaquinone which in turn condenses to melanin; an α-galactosidase, which will turn a chromogenic α-galactose substrate. Included within the terms “selectable or screenable marker genes” are also genes which encode a secretable marker whose secretion can be detected as a means of identifying or selecting for transformed cells. Examples include markers which encode a secretable antigen that can be identified by antibody interaction, or even secretable enzymes which can be detected catalytically. Secretable proteins fall into a number of classes, including small, diffusible proteins detectable, e.g., by ELISA, small active enzymes detectable in extracellular solution (e.g., α-amylase, β-lactamase, phosphinothricin transferase), or proteins which are inserted or trapped in the cell wall (such as proteins which include a leader sequence such as that found in the expression unit of extension or tobacco PR-S). Other possible selectable and/or screenable marker genes will be apparent to those of skill in the art.

Technology for introduction of DNA into cells is well known to those of skill in the art. Four general methods for delivering a gene into cells have been described: (1) chemical methods, (2) physical methods such as microinjection and bombardment, (3) viral vectors and (4) receptor-mediated mechanisms.

It is also to be understood that two different transgenic plants can also be mated to produce offspring that contain two independently segregating added, exogenous genes.

The present invention also provides for parts of the plants of the present invention. Plant parts, without limitation, include seed, endosperm, ovule and pollen. In a particularly preferred embodiment of the present invention, the plant part is a seed.

Transformation of plant protoplasts can be achieved using methods based on calcium phosphate precipitation, polyethylene glycol treatment, electroporation, and combinations of these treatments.

Any of the nucleic acid molecules of the present invention may be introduced into a plant cell in a permanent or transient manner in combination with other genetic elements such as vectors, promoters enhancers etc. Further any of the nucleic acid molecules encoding an E. nidulans protein or fragment thereof or homologs of the present invention may be introduced into a plant cell in a manner that allows for over expression of the protein or fragment thereof encoded by the nucleic acid molecule.

Uses of the Agents of the Present Invention

Nucleic acid molecules of the present invention may be employed to obtain other E. nidulans nucleic acid molecules. Such molecules can be readily obtained by using the above-described nucleic acid molecules to screen E. nidulans libraries.

Nucleic acid molecules and fragments thereof of the present invention may also be employed to obtain nucleic acid molecule homologs of non-E. nidulans species including the nucleic acid molecules that encode, in whole or in part, protein homologs of other species or other organisms, sequences of genetic elements such as promoters and transcriptional regulatory elements. Such molecules can be readily obtained by using the above-described nucleic acid molecules to screen cDNA or genomic libraries of non-E. nidulans species. Methods for forming such libraries are well known in the art. Such homolog molecules may differ in their nucleotide sequences from those found in one or more of the E. nidulans genes of this invention or complements thereof because complete complementarity is not needed for stable hybridization. The nucleic acid molecules of the present invention therefore also include molecules that, although capable of specifically hybridizing with the nucleic acid molecules may lack “complete complementarity.”

The disclosed nucleic acid molecules may be used to define one or more primer pairs that can be used with the polymerase chain reaction to amplify and obtain any desired nucleic acid molecule or fragment thereof. Such molecules will find particular use in generation of nucleic acid arrays, including microarrays, containing portions of or the entire encoding region for the identified E. nidulans genes. It is noted that the molecules on such arrays may contain native intervening sequences (introns) of the genes and will still find use in microarray based methods such as transcriptional profiling for functional analysis of E. nidulans genes and metabolic pathways. Particularly preferred primers are those set forth in table 3.

The nucleic acid molecules of the present invention may be used for physical mapping. Physical mapping, in conjunction with linkage analysis, can enable the isolation of genes. Physical mapping has been reported to identify the markers closest in terms of genetic recombination to a gene target for cloning. Once a DNA marker is linked to a gene of interest, the chromosome walking technique can be used to find the genes via overlapping clones. For chromosome walking, random molecular markers or established molecular linkage maps are used to conduct a search to localize the gene adjacent to one or more markers. A chromosome walk is then initiated from the closest linked marker. Starting from the selected clones, labeled probes specific for the ends of the insert DNA are synthesized and used as probes in hybridizations against a representative library. Clones hybridizing with one of the probes are picked and serve as templates for the synthesis of new probes; by subsequent analysis, contigs are produced.

The degree of overlap of the hybridizing clones used to produce a contig can be determined by comparative restriction analysis. Comparative restriction analysis can be carried out in different ways all of which exploit the same principle; two clones of a library are very likely to overlap if they contain a limited number of restriction sites for one or more restriction endonucleases located at the same distance from each other. The most frequently used procedures are, fingerprinting, restriction fragment mapping or the “landmarking” technique. It is understood that the nucleic acid molecules of the present invention may in one embodiment be used in physical mapping. In a preferred embodiment, nucleic acid molecules of the present invention may in one embodiment be used in the physical mapping of E. nidulans.

Nucleic acid molecules of the present invention can be used in comparative mapping. Comparative mapping within families provides a method to assess the degree of sequence conservation, gene order, ploidy of species, ancestral relationships and the rates at which individual genomes are evolving. Comparative mapping has been carried out by cross-hybridizing molecular markers across species within a given family. As in genetic mapping, molecular markers are needed but instead of direct hybridization to mapping filters, the markers are used to select large insert clones from a total genomic DNA library of a related species. The selected clones, each a representative of a single marker, can then be used to physically map the region in the target species. The advantage of this method for comparative mapping is that no mapping population or linkage map of the target species is needed and the clones may also be used in other closely related species. By comparing the results obtained by genetic mapping in model organisms, with those from other species, similarities of genomic structure among species can be established. Cross-hybridization of RFLP markers has been reported and conserved gene order has been established in many studies. Such macroscopic synteny is utilized for the estimation of correspondence of loci among these organisms. It is understood that nuclear acid molecules of the present invention may in another embodiment be used in comparative mapping. In a preferred embodiment the nucleic acid molecules of present invention may be used in the comparative mapping of filamentous fungi.

In an aspect of the present invention, one or more of the agents of the present invention may be used to detecting the presence, absence or level of a organism, preferably a filamentous fungus and more preferably an E. nidulans in a sample. In another aspect of the present invention, one or more of the nucleic acid molecules of the present invention are used to determine the level (i.e., the concentration of mRNA in a sample, etc.) or pattern (i.e., the kinetics of expression, rate of decomposition, stability profile, etc.) of the expression of a protein encoded in part or whole by one or more of the nucleic acid molecule of the present invention (collectively, the “Expression Response” of a cell or tissue). As used herein, the Expression Response manifested by a cell or tissue is said to be “altered” if it differs from the Expression Response of cells or tissues of organisms not exhibiting the phenotype. To determine whether a Expression Response is altered, the Expression Response manifested by the cell or tissue of the organism exhibiting the phenotype is compared with that of a similar cell or tissue sample of a organism not exhibiting the phenotype. As will be appreciated, it is not necessary to re-determine the Expression Response of the cell or tissue sample of organisms not exhibiting the phenotype each time such a comparison is made; rather, the Expression Response of a particular organism may be compared with previously obtained values of normal organism. As used herein, the phenotype of the organism is any of one or more characteristics of an organism.

Nucleic acid molecules of the present invention can be used to monitor expression. A microarray-based method for high-throughput monitoring of gene expression may be utilized to measure gene-specific hybridization targets. This ‘chip’-based approach involves using microarrays of nucleic acid molecules as gene-specific hybridization targets to quantitatively measure expression of the corresponding genes. Every nucleotide in a large sequence can be queried at the same time. Hybridization can be used to efficiently analyze nucleotide sequences.

Several methods have been described for fabricating microarrays of nucleic acid molecules and using such microarrays in detecting nucleic acid sequences. For instance, microarrays can be fabricated by spotting nucleic acid molecules, e.g. genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Useful substrates for arrays include nylon, glass and silicon. See, for instance, U.S. Pat. Nos. 5,202,231; 5,445,934; 5,525,464; 5,700,637; 5,744,305; 5,800,992, the entirety of the disclosures of all of which are incorporated herein by reference. Sequences can be efficiently analyzed by hybridization to a large set of oligonucleotides or cDNA molecules representing a large portion of a the genes of a genome. An array consisting of oligonucleotides or cDNA molecules complementary to subsequences of a target sequence can be used to determine the identity of a target sequence, measure its amount, and detect differences between the target and a reference sequence. Nucleic acid molecule microarrays may also be screened with molecules or fragments thereof to determine nucleic acid molecules that specifically bind molecules or fragments thereof.

The microarray approach may also be used with polypeptide targets (U.S. Pat. No. 5,445,934; U.S. Pat. No. 5,143,854; U.S. Pat. No. 5,079,600; U.S. Pat. No. 4,923,901, all of which are herein incorporated by reference in their entirety). Essentially, polypeptides are synthesized on a substrate (microarray) and these polypeptides can be screened with either protein molecules or fragments thereof or nucleic acid molecules in order to screen for either protein molecules or fragments thereof or nucleic acid molecules that specifically bind the target polypeptides.

It is understood that one or more of the molecules of the present invention, preferably one or more of the nucleic acid molecules or protein molecules or fragments thereof of the present invention may be utilized in a microarray based method. In a preferred embodiment of the present invention, one or more of the E. nidulans nucleic acid molecules or protein molecules or fragments thereof of the present invention may be utilized in a microarray based method. A particular preferred microarray embodiment of the present invention is a microarray comprising nucleic acid molecules encoding genes or fragments thereof that are homologs of known genes or nucleic acid molecules that comprise genes or fragments thereof that elicit only limited or no matches to known genes. A further preferred microarray embodiment of the present invention is a microarray comprising nucleic acid molecules having genes or fragments thereof that are homologs of known genes and nucleic acid molecules that comprise genes or fragment thereof that elicit only limited or no matches to known genes.

In a preferred embodiment, the microarray of the present invention comprises at least 10 nucleic acid molecules that specifically hybridize under high stringency to at least 10 nucleic acid molecules encoding E. nidulans protein or fragments. In a more preferred embodiment, the microarray of the present invention comprises at least 100 nucleic acid molecules that specifically hybridize under high stringency to at least 100 nucleic acid molecules that encode an E. nidulans protein or fragment thereof. In an even more preferred embodiment, the microarray of the present invention comprises at least 1,000 nucleic acid molecules that specifically hybridize under high stringency to at least 1,000 nucleic acid molecules that encode an E. nidulans protein or fragment thereof. In a further even more preferred embodiment, the microarray of the present invention comprises at least 2,500 nucleic acid molecules that specifically hybridize under high stringency to at least 2,500 nucleic acid molecules that encode an E. nidulans protein or fragment thereof. While it is understood that a single nucleic acid molecule may encode more than one protein or fragment thereof, in a preferred embodiment, at least 50%, preferably at least 70%, more preferably at least 80%, even more preferably at least 90% of the nucleic acid molecules that comprise the microarray encode one protein homolog or fragment thereof. It is, of course, understood that these nucleic acid molecules can be non-identical.

In a preferred embodiment, the microarray of the present invention comprises at least 10 nucleic acid molecules that specifically hybridize under high stringency to at least 10 ENUs selected from the group having SEQ ID NO: 16207 through SEQ ID NO: 28905 or fragment thereof or complement of either. In a more preferred embodiment, the microarray of the present invention comprises at least 100 nucleic acid molecules that specifically hybridize under high stringency to at least 100 ENUs selected from the group having SEQ ID NO: 16207 through SEQ ID NO: 28905 or fragment thereof or complement of either. In an even more preferred embodiment, the microarray of the present invention comprises at least 1,000 nucleic acid molecules that specifically hybridize under high stringency to at least 1,000 ENUs selected from the group having SEQ ID NO: 16207 through SEQ ID NO: 28905 or fragment thereof or complement of either. In a further even more preferred embodiment, the microarray of the present invention comprises at least 2,500 nucleic acid molecules that specifically hybridize under high stringency to at least 2,500 ENUs selected from the group having SEQ ID NO: 16207 through SEQ ID NO: 28905 or fragment thereof or complement of either. While it is understood that a single nucleic acid molecule may encode more than one protein homolog or fragment thereof, in a preferred embodiment, at least 50%, preferably at least 70%, more preferably at least 80%, even more preferably at least 90% of the nucleic acid molecules that comprise the microarray encode one protein or fragment thereof.

Nucleic acid molecules of the present invention may be used in site directed mutagenesis. Site-directed mutagenesis may be utilized to modify nucleic acid sequences, particularly as it is a technique that allows one or more of the amino acids encoded by a nucleic acid molecule to be altered (e.g. a threonine to be replaced by a methionine). Three basic methods for site-directed mutagenesis are often employed, i.e. (a) cassette mutagenesis, (b) primer extension and (c) methods based on PCR. See also U.S. Pat. No. 5,880,275, U.S. Pat. No. 5,380,831, and U.S. Pat. No. 5,625,136, the entirety of all of which is incorporated herein by reference.

Any of the nucleic acid molecules of the present invention may either be modified by site-directed mutagenesis or used as, for example, nucleic acid molecules that are used to target other nucleic acid molecules for modification. It is understood that mutants with more than one altered nucleotide can be constructed using techniques that practitioners skilled in the art are familiar with such as isolating restriction fragments and ligating such fragments into an expression vector.

Preferred aspects of this invention comprise collections of genes, nucleic acid molecules, polypeptides and/or primers of this invention ranging in size from about 10 non-identical members or more, e.g. at least about 100 or 270 or higher, more preferably at least about 300 or 350, most preferably at least 500 or higher, up to about 1000, or 2000 or even higher, say about 5000, or more non-identical members. As used herein a non-identical member is a member that differs in nucleic acid or amino acid sequence. For example, a non-identical nucleic acid molecule is a nucleic acid molecule that differs in nucleic acid sequence from the nucleic acid molecule to which it is being compared to. For example a nucleic acid molecule having the sequence 5′ CCC 3′ is not identical—i.e. non-identical—to a nucleic acid molecule having the sequence 5′ CCG 3′. In one aspect a collection may comprise all of the genes, nucleic acid molecules, polypeptides and/or primers of this invention. Such collections can be located or organized in a variety of forms, e.g. on microarrays, in solutions, in bacterial clone libraries, etc. As used herein, an “organized” collection is a collection where the nucleic acid or amino acid sequence of a member of such a collection can be determined based on its physical location.

Preferred collections of nucleic acid molecules can be selected from the following groups: SEQ ID NO: 16207 through SEQ ID NO: 27905 or complements thereof; SEQ ID NO: 16207 through SEQ ID NO: 26804 or complements thereof; SEQ ID NO: 26000 through SEQ ID NO: 26804 or complements thereof; SEQ ID NO: 16207 through SEQ ID NO: 25999 or complements thereof; SEQ ID NO: 24035 through SEQ ID NO: 25999 or complements thereof; SEQ ID NO: 16207 through SEQ ID NO: 24034 or complements thereof; SEQ ID NO: 22710 through SEQ ID NO: 24034 or complements thereof; SEQ ID NO: 16207 through SEQ ID NO: 22709 or complements thereof; SEQ ID NO: 17681 through SEQ ID NO: 22709 or complements thereof; SEQ ID NO: 16207 through SEQ ID NO: 17680 or complements thereof; SEQ ID NO: 17618 through SEQ ID NO: 17680 or complements thereof; SEQ ID NO: 16207 through SEQ ID NO: 17617 or complements thereof; SEQ ID NO: 17295 through SEQ ID NO: 17617 or complements thereof; SEQ ID NO: 16207 through SEQ ID NO: 17294 or complements thereof; SEQ ID NO: 28166 through SEQ ID NO: 44345 or complements thereof. Other preferred nucleic acid collections include any of the above groups but where such groups also include fragments of such sequences.

It is understood that all these preferred collections may also range in size from about 10 or more, e.g. at least about 100 or 270 or higher, more preferably at least about 300 or 350, most preferably at least 500 or higher, up to about 1000, or 2000 or even higher, say about 5000, or more non-identical members.

Another aspect of this invention provides the genes, nucleic acid molecules, polypeptides and/or primers in a substantially pure form. For instance, by use of the primers of this invention, any of the ENUs can be produced in substantially pure form by PCR.

Another aspect of this invention is to provide methods for determining gene expression, e.g. identifying homologous genes expressed by non-E. nidulans organism. Such methods comprise collecting mRNA from tissue of such organism, using the mRNA as a template for producing a quantity of labeled nucleic acid, and contacting the labeled nucleic acid molecule with a collection of purified nucleic acid molecules, e.g. on a microarray.

Computer Media

One or more of the nucleotide sequence provided in SEQ ID NO: 1, through SEQ ID NO: 44345 or complements or fragments of either can be “provided” in a variety of media to facilitate use. Such a medium can also provide a subset thereof in a form that allows a skilled artisan to examine the sequences. In one application of this embodiment, a nucleotide sequence of the present invention can be recorded on computer readable media. As used herein, “computer readable media” refers to any medium that can be read and accessed directly by a computer. Such media include, but are not limited to: magnetic storage media, such as floppy discs, hard disc, storage medium, and magnetic tape: optical storage media such as CD-ROM; electrical storage media such as RAM and ROM; optical scanner readable medium such as printed paper; and hybrids of these categories such as magnetic/optical storage media. A skilled artisan can readily appreciate how any of the presently known computer readable mediums can be used to create a manufacture comprising computer readable medium having recorded thereon a nucleotide sequence of the present invention.

As used herein, “recorded” refers to a process for storing information on computer readable medium. A skilled artisan can readily adopt any of the presently known methods for recording information on computer readable medium to generate media comprising the nucleotide sequence information of the present invention. In addition, a variety of data processor programs and formats can be used to store the nucleotide sequence information of the present invention on computer readable medium. The sequence information can be represented in a word processing text file, or represented in the form of an ASCII file, stored in a database application, such as DB2, Sybase, Oracle, or the like. A skilled artisan can readily adapt any number of data processor structuring formats (e.g. text file or database) in order to obtain computer readable medium having recorded thereon the nucleotide sequence information of the present invention.

By providing one or more of nucleotide sequences of the present invention, a skilled artisan can routinely access the sequence information for a variety of purposes. Computer software is publicly available which allows a skilled artisan to access sequence information provided in a computer readable medium. The examples which follow demonstrate how software which implements the BLAST and/or BLAZE search algorithms on a Sybase system can be used to identify open reading frames (ORFs) within the genome that contain homology to ORFs or proteins from other organisms. Such ORFs are protein-encoding fragments within the sequences of the present invention and are useful in producing commercially important proteins such as enzymes used in amino acid biosynthesis, metabolism, transcription, translation, RNA processing, nucleic acid and a protein degradation, protein modification, and DNA replication, restriction, modification, recombination, and repair.

The present invention further provides systems, particularly computer-based systems, which contain the sequence information described herein. Such systems are designed to identify commercially important fragments of the nucleic acid molecule of the present invention. As used herein, “a computer-based system” refers to the hardware means, software means, and data storage means used to analyze the nucleotide sequence information of the present invention. The minimum hardware means of the computer-based systems of the present invention comprises a central processing unit (CPU), input means, output means, and data storage means. A skilled artisan can readily appreciate that any one of the currently available computer-based system are suitable for use in the present invention.

As indicated above, the computer-based systems of the present invention comprise a data storage means having stored therein a nucleotide sequence of the present invention and the necessary hardware means and software means for supporting and implementing a search means. As used herein, “data storage means” refers to memory that can store nucleotide sequence information of the present invention, or a memory access means which can access manufactures having recorded thereon the nucleotide sequence information of the present invention. As used herein, “search means” refers to one or more programs which are implemented on the computer-based system to compare a target sequence or target structural motif with the sequence information stored within the data storage means. Search means are used to identify fragments or regions of the sequence of the present invention that match a particular target sequence or target motif. A variety of known algorithms are disclosed publicly and a variety of commercially available software for conducting search means are available can be used in the computer-based systems of the present invention. Examples of such software include, but are not limited to, MacPattern (EMBL), BLASTIN and BLASTIX (NCBIA). One of the available algorithms or implementing software packages for conducting homology searches can be adapted for use in the present computer-based systems.

The most preferred sequence length of a target sequence is from about 30 to 300 nucleotide residues or from about 10 to 100 of the corresponding amino acids. However, it is well recognized that during searches for commercially important fragments of the nucleic acid molecules of the present invention, such as sequence fragments involved in gene expression and protein processing, may be of shorter length.

As used herein, “a target structural motif,” or “target motif,” refers to any rationally selected sequence or combination of sequences in which the sequences the sequence(s) are chosen based on a three-dimensional configuration which is formed upon the folding of the target motif. There are a variety of target motifs known in the art. Protein target motifs include, but are not limited to, enzymatic active sites and signal sequences. Nucleic acid target motifs include, but are not limited to, promoter sequences, cis elements, hairpin structures and inducible expression elements (protein binding sequences).

Thus, the present invention further provides an input means for receiving a target sequence, a data storage means for storing the target sequences of the present invention sequence identified using a search means as described above, and an output means for outputting the identified homologous sequences. A variety of structural formats for the input and output means can be used to input and output information in the computer-based systems of the present invention. A preferred format for an output means ranks fragments of the sequence of the present invention by varying degrees of homology to the target sequence or target motif. Such presentation provides a skilled artisan with a ranking of sequences which contain various amounts of the target sequence or target motif and identifies the degree of homology contained in the identified fragment.

Having now generally described the invention, the same will be more readily understood through reference to the following examples which are provided by way of illustration, and are not intended to be limiting of the present invention, unless specified.

### EXAMPLE 1

This example serves to illustrate the generation of the 16206 nucleic acid sequences listed in Table 1 as contigs having SEQ ID NO: 1 through SEQ ID NO: 16206. About 390,000 genomic nucleotide sequence traces are derived from 11 different M13 and double stranded libraries. The two basic methods for the DNA sequencing are the chain termination method of Sanger et al., Proc. Natl. Acad. Sci. (U.S.A.) 74:5463-5467 (1977) and the chemical degradation method of Maxam and Gilbert, Proc. Natl. Acad. Sci. (U.S.A.) 74:560-564 (1977) using automated fluorescence-based sequencing as reported by Craxton, Method, 2:20-26 (1991); Ju et al., Proc. Natl. Acad. Sci. (U.S.A.) 92:4347-4351 (1995); and Tabor and Richardson, Proc. Natl. Acad. Sci. (U.S.A.) 92:6339-6343 (1995) and high speed capillary gel electrophoresis, e.g. as disclosed by Swerdlow and Gesteland, Nucleic Acids Res. 18:1415-1419 (1990); Smith, Nature 349:812-813 (1991); Luckey et al., Methods Enzymol. 218:154-172 (1993); Lu et al., J. Chromatog. A. 680:497-501 (1994); Carson et al., Anal. Chem. 65:3219-3226 (1993); Huang et al., Anal. Chem. 64:2149-2154 (1992); Kheterpal et al., Electrophoresis 17:1852-1859 (1996); Quesada and Zhang, Electrophoresis 17:1841-1851 (1996); Baba, Yakugaku Zasshi 117:265-281 (1997). For instance, genomic nucleotide sequence traces are generated using a 377 DNA Sequencer (Perkin-Elmer Corp., Applied Biosystems Div., Foster City, Calif.) allowing for rapid electrophoresis and data collection. With these types of automated systems, fluorescent dye-labeled sequence reaction products are detected and chromatograms are subsequently viewed, stored in computer and analyzed using corresponding apparatus-related software programs. These methods are known to those of skill in the art and have been described and reviewed (Birren et al., Genome Analysis: Analyzing DNA, 1, Cold Spring Harbor, N.Y.

Over 390,000 quality genomic sequence traces are assembled generally as follows:
(a) all traces are quality clipped using yc_qual_clip.pl (with a minimum PHRED score of 12.5 and maximum length of 50 bp);
(b) all traces are segregated according to library construction method;
(c) all traces are “vector-trimmed’ i.e., 5′ and 3′ vector and linker sequences are removed;
(d) all traces are re-united in one file;
(e) all traces are then clustered with PANGEA's clustering tool (available from Pangea Corp., Pittsburgh, Pa.). A cluster includes 2 or more traces of sequences with 90% similarity over 60 bp. After clustering the set of traces includes clusters and non-clustered traces referred to as “singletons”.
(f) A high stringency PHRAP assembly is run on each cluster to separate from clusters singlet traces which do not meet stringency criteria. The arguments to high stringency PHRAP are: minmatch 25, minscore 50, penalty −4;
(g) Contigs and the singleton (including singlet) traces and their corresponding quality files are united; and, then are assembled with a low stringency PHRAP (using default PHRAP arguments) to generate a “final” assembly; and
(h) the final set of 16,144 nucleic acid sequences (identified in Table 1 by contig identification number “ANI61xxxx” and by the corresponding SEQ ID NO: 1 through SEQ ID NO:16144) and 52 nucleic acid sequences (identified in Table 1 by contig identification number “ANI50xxxx” and by corresponding SEQ ID NO: 16145 through SEQ ID NO:16206) are run through the annotation and gene selection processes. Contigs in SEQ ID NO:1 through SEQ ID NO:16144 are recognized as those sequences whose designations begin with ANI61C or ANI50C. Singleton sequences are recognized as those having designations which begin with ANI61S or ANI50S.
   The genomic sequence traces and many of the contigs and singleton traces are disclosed in copending provisional applications for patent identified by Ser. Nos. 60/101,665; 60/101,666; 60/102,358; 60/113,361; 60/126,265; 60/130,189; 60/130,190; 60/132,861; 60/138,103; 60/149,882.

### EXAMPLE 2

This example illustrates the identification of ENUs within 16206 contigs assembled in Example 1. The genes and partial genes embedded in such contigs are identified through a series of informatic analyses. The tools to define genes fall into two categories: homology-based and predictive-based methods. Homology-based searches (e.g., GAP2, NAP, BLASTX and TBLASTX) detect conserved sequences during comparisons of DNA sequences or hypothetically translated protein sequences to public and/or proprietary DNA and protein databases. Existence of a E. nidulans gene is inferred if significant sequence similarity extends over the majority of the target gene. Since homology-based methods may overlook genes unique to E. nidulans, for which homologous nucleic acid molecules have not yet been identified in databases, gene prediction programs are also used. Predictive methods employed in the definition of the E. nidulans genes included the use of the GenScan gene predictive software program which is available from Stanford University (e.g. at the web site http://gnomic/stanford.edu/GENSCANW.html). GenScan, in general terms, infers the presence and extent of a gene through a search for “gene-like” grammar.

The homology-based methods used to define the E. nidulans gene set included GAP2, BLASTX supplemented by NAP, and TBLASTX. For a description of BLASTX and TBLASTX see Coulson, Trends in Biotechnology 12:76-80 (1994) and Birren et al., Genome Analysis, 1:543-559 (1997). GAP2 and NAP are part of the Analyis and Annotation Tool (AAT) for Finding Genes in Genomic Sequences which was developed by Xiaoqiu Huang at Michigan Tech University and is available at the web site http://genome.cs.mtu.edu/. The AAT package includes two sets of programs, one set (DPS/NAP) for comparing the query sequence with a protein database, and the other set (DDS/GAP2) for comparing the query sequence with a cDNA database. Each set contains a fast database search program and a rigorous alignment program. The database search program quickly identifies regions of the query sequence that are similar to a database sequence. Then the alignment program constructs an optimal alignment for each region and the database sequence. The alignment program also reports the coordinates of exons in the query sequence. See Huang, et al., Genomics 46: 37-45 (1997).

The GAP2 program computes an optimal global alignment of a genomic sequence and a cDNA sequence without penalizing terminal gaps. A long gap in the cDNA sequence is given a constant penalty. The DNA-DNA alignment by GAP2 adjusts penalties to accommodate introns. The GAP2 program makes use of splice site consensuses in alignment computation. GAP2 delivers the alignment in linear space, so long sequences can be aligned. See Huang, Computer Applications in the Biosciences 10 227-235 (1994). The GAP2 program aligned the E. nidulans contigs with the A. nidulans/E. nidulans EST library in the microorganism databank maintained by Bruce Roe's laboratory at the University of Oklahoma.

The NAP program computes a global alignment of a DNA sequence and a protein sequence without penalizing terminal gaps. NAP handles frameshifts and long introns in the DNA sequence. The program delivers the alignment in linear space, so long sequences can be aligned. It makes use of splice site consensuses in alignment computation. Both strands of the DNA sequence are compared with the protein sequence and one of the two alignments with the larger score is reported. See Huang, and Zhang, “Computer Applications in the Biosciences 12(6), 497-506 (1996).

NAP takes a nucleotide sequence, translates it in three forward reading frames and three reverse complement reading frames, and then compares the six translations against a protein sequence database (e.g. the non-redundant protein (i.e., nr-aa) database maintained by the National Center for Biotechnology Information as part of GenBank and available at the web site: http://www.ncbi.nlm.nih.gov). TBLASTX compared six possible frame translations of the E. nidulans contigs against six frame translations of Aspergillus fumigatus, Fusarium gramineareum, Saccharomyces cerevisiae, and Candida albicans genomic sequences.

The first homology-based search for genes in the E. nidulans contigs is effected using the GAP2 program and the University of Oklahoma A. nidulans/E. nidulans EST database. A collection of about 14000 A. nidulans/E. nidulans EST sequences from the database with known 5′ and 3′ orientations and mate information are clustered into about 3500 distinct sets or “clusters”. These clusters are then mapped onto an assembly of E nidulans contigs represented by SEQ ID NO. 1 through SEQ ID NO. 16206 using the GAP2 program. GAP2 standards for selecting a DNA-DNA match were >96% sequence identity with the following parameters:

gap extension penalty=1

match score=2

gap open penalty=6

gap length for constant penalty=20

mismatch penalty=−2

minimum exon length=21

DNA matches with ESTs fell into three categories. Firstly, ENUs are identified when a 5′-3′ EST pair aligned to the sequences on the same contig. Since EST's are necessarily derived from genes, no corroborating evidence is required to validate the gene prediction. Certain ENUs are identified by 5′-3′ EST pair match on a single contig. These ENUs are identified by “EST” in the selection basis column of Table 2 and include SEQ ID NO. 16207 through SEQ ID NO. 17294.

Another group of ENUs identified by DNA match with EST's is selected because of alignment of a 5′-3′ EST pair which spanned two contigs supported by BLASTX similarity or clonemate information. These ENUs are identified by “MCEST” in the selection basis column of Table 2 and include SEQ ID NO. 17618 through SEQ ID NO. 17680.

Another group of ENUs identified by DNA match with EST's is selected solely from a 3′ EST match of at least 300 bp using EST's which are not previously aligned. These ENUs are identified by “TPEST” in the selection basis column of Table 2 and include SEQ ID NO. 17295 through SEQ IS NO. 17617.

The second homology-based method used for gene discovery is BLASTX hits extended with the NAP software package. BLASTX is run with the E. nidulans contigs represented by SEQ ID NO. 1 through SEQ ID NO. 16206 as queries against the GenBank non-redundant protein data library identified as “nr-aa”. NAP is used to better align the amino acid sequences as compared to the genomic sequence. NAP extends the match in regions where BLASTX has identified high-scoring-pairs (HSPs), predicts introns, and then links the exons into a single ORF prediction. Experience suggests that NAP tends to mis-predict the first exon. E. nidulans introns are almost without exception short (<150 bp), and NAP routinely predicts very long (>400 bp) introns leading to a very short, and biologically unmeaningful, 5′ exon. The NAP-predicted ORFs containing long introns (>175 bp) are first segregated and truncated (the long intron and the nonsense 5′ exon removed) and the remaining portion of the ORF established as a gene. Selection in a first pass is for sequences with (a)<600 bp from the 3′ end with >50% coverage, (b)<600 bp from the 3′ end with >300 bp coverage and (c)>1000 bp from the 3′ end with 500 bp coverage. Selection in a second pass is for sequences with (a)<300 bp from the 3′ end with, 500 bp coverage and >80% coverage or (b)<300 bp from the 3′ end and >500 bp coverage. The NAP parameters are:

gap extension penalty=1

gap open penalty=15

gap length for constant penalty=25

min exon length (in aa)=7

The ENUs identified by NAP with (a)>300 bp and >10% homology or (b)>175 bp and >50% coverage are identified by “NAP” in the selection basis column of Table 2 and include SEQ ID NO. 17681 through SEQ ID NO. 22709.

For NAP alignments with large introns GenScan are used to locate the terminal exon and extend the 5′ end of the terminal exon. When there is no GenScan indication of a terminal exon, the gene is identified using the longest exon cluster without a large intron. The ENUs identified from large intron alignments are identified by “LINAP” in the selection basis column of Table 2 and include SEQ ID NO. 22710 through SEQ ID NO. 24034.

In the final homology-based method, TBLASTX, is used with genome information from three fungal genome sequencing projects: Aspergillus fumigatus, Fusarium gramineareum, Saccharomyces cerevisiae and Candida albicans. As a general rule, non-coding regions of DNA accumulate mutations much more rapidly than coding regions. With this knowledge, we use TBLASTX, which compares hypothetical translations, to identify regions of DNA that code for highly similar amino acid strings in both E. nidulans and the four other fungal genomes. As with EST matches, the TBLASTX hits fall into three categories of defined genes: matches that fall within an E. nidulans contig, matches that convincingly bridge contigs, and long matches that contain sufficient portions of a gene for use in transcriptional profiling. Unlike GAP2 and BLASTX/NAP analyses, we have comparatively little experience in interpreting TBLASTX scores as a tool for defining the unigene set. For this reason, conservative standards for inclusion of TBLASTX hits into the gene set are utilized. These standards are a minimal E value of 1E-20, and for terminal exons, a minimal match of 200 bp within the 1000 most 5′ and 3′ ends of an E. nidulans contig. In addition to these criteria, in part due to conflicting data from TBLASTX analyses (where different TBLASTX matches will suggest two or more mutually exclusive possibilities) and to concerns that repeat regions may be sufficiently similar to confound the method, TBLASTX predicted genes bridging two contigs are included when corroborating evidence in the form of GenScan predictions and/or clone mate evidence from double stranded clones is available.

The GenScan program is “trained” with E. nidulans characteristics. Though better than the “off-the-shelf” version, the GenScan trained to identify E. nidulans genes proved more proficient at predicting exons than predicting full-length genes. Predicting full-length genes is compromised by point mutations in the unfinished contigs, as well as by the short length of the contigs relative to the typical length of a gene. Due to the errors found in the full-length gene predictions by GenScan, inclusion of GenScan-predicted genes is limited to those genes and exons whose probabilities are above a conservative probability threshold. When used with TBLASTX the GenScan parameters are:

mean GenScan P value >0.3

mean GenScan T value >0

mean GenScan Coding score >50

length >200 bp

minimum TBLASTX E value <1E-20

Significant TBLASTX hits to single contigs that are greater than 300 bp contributed 805 genes to the unigene set. The high E value threshold limited the vast majority (99%) of the TBLASTX hits to the fungal genome comparisons. The TBLASTX hits with GenScan corroboration identified 1965 ENUs identified by “GTBX” in the selection basis column of Table 2 and include SEQ ID NO. 24035 through SEQ ID NO. 25999.

To identify ENUs solely by TBLASTX, the TBASTX E values is set at 1E-30 with a length of >200 bp. The ENU's identified solely by TBLASTX are identified by “TBX” in the selection basis column of Table 2 and include SEQ ID NO. 26000 through SEQ ID NO. 26804.

A final set of genes is predicted using the GenScan program “trained” with E. nidulans characteristics and the mean GenScan P value parameters changed to >0.4. The ENUs identified solely by GenScan are identified by “GSP” in the selection basis column of Table 2 and include SEQ ID NO. 26805 through SEQ ID NO. 27905.

To insure that the same nucleic acid molecule is not inferred two or more times with different methods, an all-versus-all BLASTN analysis of the all the identified ENUs is conducted. There are instances where sequencing and assembly errors will confound the identification of duplicates, but such instances are comparatively rare.

The confidence in accuracy of the identified ENUs is highest for those identified by a match of a 5′-3′ EST to a single contig (identified by EST) and lowest for those identified solely the GenScan predictive algorithm (identified by GSP). The order of confidence for the ENUs is in the following order:

| Selection Basis | Confidence |
|---|---|
| EST | highest |
| TPEST | |
| MCEST | |
| NAP | |
| LINAP | |
| GTBX | |
| TBX | |
| GSP | lowest |

In Table 2 the ENUs of this invention are identified in the sequence identification (seq. id.) column the name ENU (Emericella nidulans unigene) and begins with ENU00001 for SEQ ID NO. 16207.

Other modifications of the above described embodiments of the invention which are obvious to those of skill in the area of molecular biology and related disciplines are intended to be within the scope of the following claims.

### EXAMPLE 3

This example serves to illustrate the design of primers of this invention which are useful, for instance, for initiating synthesis of nucleic acid molecules of this invention, specifically substantial parts of certain ENU's of this invention. The primers specifically disclosed herein, i.e. in Table 3 by SEQ ID NO. 28166 through SEQ ID NO. 44345, are designed with the program Primer3 (obtained from the MIT-Whitehead Genome Center) with a “perl-oracle” wrapper. The criteria applied to design a primer included:

Primer annealing temperature (minimum 65° C., optimum 70° C., maximum 75° C.)

Primer length (minimum 18 bp, optimum 20 bp, maximum 28 bp)

G+C content (minimum 20%, maximum 80%)

Position of the primer relative to the gene

Length of the amplified region (500 to 800 bp)

PHRED quality score of the gene template (minimum of 20)

Whether the gene was defined from one or two contigs

Maximum mismatch=12.0 (weighted score from Primer3 program)

Pair Max Misprime=24.0 (weighted score from Primer3 program)

Maximum N's=0

Maximum poly-X=5

The primary goal of the design process is the creation of groups of primer pairs with a common annealing temperature (Tₘ). When the program could identify a primer pair for any gene that fit the criteria, the gene is removed from the bin of genes needing primer design. Genes remaining in the bin are subjected to additional rounds of primer-picking, with the gradual and simultaneous relaxation of the criteria (i.e., lowering the annealing temperature, increasing the size of the window where primers could be predicted, expanding the range of permitted size and G+C content, removing the need for a G/C clamp), until primers are picked for about 8,000 of the about 12,000 ENUs of this invention. After the E. nidulans specific portion of the primers is selected, an additional common primer tail sequence (universal primer) is added to the 5′ ends. For the forward primers, the additional common bases added are: (5′-GAATTCACTGCGGCCGCCATG-3′); for the reverse primers the additional common bases added are: (5′-GTTCTCGAGACGAGCGATCGC-3′). The universal primer tail sequences are added so that subsequent reamplifications of any primer pair can be done with a single set of primers. In addition, the primer tail sequences contain restriction digestion sites for 8 bp cutters (NotI and SgfI) and 6 bp cutters (EcoRI and XhoI) to facilitate cloning of ENUs into vectors. The forward primers contains EcoRI and NotI restriction sites; the reverse primers contains XhoI and SgfI restriction sites.

Reference is also made to Tables 2 and 3 for identification of the primers and reference to the ENU for which they are designed. The primer pair for a particular ENU is identified in Table 2 by indication of the complementary or identical nucleotides in the particular ENU under the columns “Primer 5 pos” and “Primer 3 pos”. The primer sequence numbers in Table 3 correspond to an ENU identified in the “Seq id” column. For example, the primer pair ENU00001p5 and ENU00001p3 represent the sequences for the 5′ and 3′ primer, respectively for ENU00001. The primer sequences provided in the sequence listing all contain the universal tail sequence described above as the first 21 nucleotides. It is noted that primer pairs are not required to contain the universal tail sequence, the relevant portion for amplification and/or hybridization probes being the E. nidulans specific sequences designated in the “Primer 5 pos” and “Primer 3 pos” columns in Table 2.

Lengthy table referenced here
US20090119022A1-20090507-T00001
Please refer to the end of the specification for access instructions.

### Table Column Heading Descriptions

### Table 1

Sea Num

Provides the SEQ ID NO. for the listed sequences.

Contig Id

Arbitrary identification assigned to each contig or singleton. Contigs designations begin with ANI61C or ANI50C. Singleton designations begin with ANI61S or ANI50S.

### Table 2

Sea Num

Provides the SEQ ID NO. for the listed sequences.

Sea Id

Arbitrarily assigned number for each ENU (Emericella nidulans unigene).

Contig Source

Indicates contigs or singletons from which the ENUs are identified and the location of the ENU within the contig or singleton. In cases where the first numeral is higher than its corresponding second numeral, the E. nidulans protein or fragment thereof is encoded by the complement of the sequence set forth in the sequence listing. The first numeral separated from the contig or singleton ID by a colon represents the starting point for the codon for the most N-terminal (if the first number is lower than the second number) or C-terminal (if the first number is higher than the second number) amino acid for the protein or protein fragment encoded by the ENU. For MCEST selected ENUs, locations on each of the overlapping contigs or contig and singleton are provided.

Primer 5 Pos

Indicates the sequence segment within the ENU which is complementary to the hybridizing portion of the 5′ or forward primer.

Primer 3 Pos

Indicates the sequence segment within the ENU which is identical to the hybridizing portion of the 3′ or reverse primer.

Selection Basis

A code which identifies the ENU selection method. The selection methods are described in detail in Example 2 and briefly summarized as follows:
EST: GAP2 identified 5′-3′ EST pair match on a single contig or singleton
TPEST: GAP2 identified 3′ EST match of at least 300 bp
MCEST: GAP2 identified 5′-3′ EST pair match spanning two contigs or a contig and a singleton
NAP: NAP predicted ORFs which have no unreasonably long introns (>175 bp)
LINAP: NAP predicted ORFs with long predicted introns and false 5′ exons removed
GTBX: TBLASTX hit with GenScan corroboration
TBX: TBLASTX hit alone
GSP: GenScan prediction
Database Hit

Indicates database entry for sequence which matched to the E. nidulans contig query. For EST and MCEST hits, the database is the University of Oklahoma A. nidulans/E. nidulans EST database. For GTBX and TBX hits, the database is a private microbial sequence database containing genomic sequences of Aspergillus fumigatus, Fusarium gramineareum, Saccharomyces cerevisiae and Candida albicans.

Ncbi Gi

Refers to National Center for Biotechnology Information GenBank Identifier number which is the best match for a given contig or singleton region from which the associated ENU was identified using the NAP or LINAP selection basis.

Aat Score

The aat_nap score is reported by the NAP program in the AAT package. It is an alignment score in which each match and mismatch is scored based on the BLOSUM62 scoring matrix.

Blast Score

Each entry in the “Blast Score” column of the table refers to the BLASTX score that is generated by sequence comparison of the designated clone with the GenBank sequence listed in the Description column.

Blast Prob

The entries in the “Blast-Prob” column refer to the probability that matches occur by chance.

% Id

The entries in the “% id” column of the table refer to the percentage of identically matched nucleotides (or residues) that exist along the length of that portion of the sequences which is aligned by the BLAST comparison portion of the NAP program.

% Cvrg

The “% cvrg” is the percent of hit sequence length that matches to the query sequence in the match generated using NAP (% cvrg=(match length/hit total length)×100).

Description

For NAP and LINAP selected ENUs, a description of the database entry referenced in the “NCBI gi” column. For EST, TPEST, and MCEST, the resulting ENU sequences were analyzed by TBLASTX against the non-redundant protein database maintained by NCBI, and a description of the top hit is provided.

### Table 3

Sea Num

Provides the SEQ ID NO. for the listed primer sequences. The first 21 nucleotides of each primer sequence contains either a universal 5′ or 3′ tail sequence.

Seq Id

Identification assigned to each primer sequence. Primers are identified by the number of the ENU and either p5 to indicate the 5′ or forward primer, or p3 to indicate the 3′ or reverse primer. The location of the E. nidulans specific sequence within the primers is provided in Table 2.

LENGTHY TABLES
The patent application contains a lengthy table section. A copy of the table is available in electronic form from the USPTO web site (http://seqdata.uspto.gov/?pageRequest=docDetail&DocID=US20090119022A1). An electronic copy of the table will also be available from the USPTO upon request and payment of the fee set forth in 37 CFR 1.19(b)(3).

1-46. (canceled)

47. A method of identifying a nucleotide sequence using a computer, said method comprising comparing a target sequence to one or more sequences stored in computer readable medium having recorded thereon at least 100 nucleotide sequences including at least one sequence selected from the group consisting of SEQ ID NO: 16207 through SEQ ID NO: 18349, SEQ ID NO: 18351 through SEQ ID NO: 18356, SEQ ID NO: 18358 through SEQ ID NO: 21010, and SEQ ID NO: 21012 through SEQ ID NO: 27905 and complements thereof, and identifying said target sequence as being present in the computer readable medium based on said comparison, wherein said target sequence is compared to at least one sequence selected from the group consisting of SEQ ID NO: 16207 through SEQ ID NO: 18349, SEQ ID NO: 18351 through SEQ ID NO: 18356, SEQ ID NO: 18358 through SEQ ID NO: 21010, and SEQ ID NO: 21012 through SEQ ID NO: 27905, and wherein at least one of said comparing and identifying steps is carried out via the computer.

48. The method according to claim 47, wherein both of said comparing and identifying steps are carried out via the computer.

49. A method for identifying a nucleic acid sequence using a computer, said method comprising:
a) providing a target nucleotide sequence;
b) comparing said target nucleotide sequence to one or more nucleotide sequences stored in a computer readable medium having recorded thereon at least 100 nucleotide sequences including at least one sequence selected from the group consisting of SEQ ID NO: 16207 through SEQ ID NO: 18349, SEQ ID NO: 18351 through SEQ ID NO: 18356, SEQ ID NO: 18358 through SEQ ID NO: 21010, and SEQ ID NO: 21012 through SEQ ID NO: 27905 and complements thereof, wherein said target nucleotide sequence is compared to at least one of said sequences selected from the group consisting of SEQ ID NO: 16207 through SEQ ID NO: 18349, SEQ ID NO: 18351 through SEQ ID NO: 18356, SEQ ID NO: 18358 through SEQ ID NO: 21010, and SEQ ID NO: 21012 through SEQ ID NO: 27905; and
c) identifying said target nucleotide sequence as having significant sequence identity to said one or more nucleotide sequences stored in a computer readable medium based on said comparison, wherein at least one of said comparing and identifying steps is carried out via the computer.

50. The method according to claim 49, wherein both of said comparing and identifying steps are carried out via the computer.

51. The method according to claim 49, wherein said target sequence shares between 100% and 90% sequence identity with one or more of said nucleotide sequences stored on a computer readable medium.

52. The method according to claim 51, wherein said target sequence shares between 100% and 95% sequence identity with one or more of said nucleotide sequences stored on a computer readable medium.

53. The method according to claim 52, wherein said target sequence shares between 100% and 98% sequence identity with one or more of said nucleotide sequences stored on a computer readable medium.

54. The method according to claim 53, wherein said target sequence shares between 100% and 99% sequence identity with one or more of said nucleotide sequences stored on a computer readable medium.

55. The method according to claim 49, wherein said target sequence is identified as homologous to an open reading frame (ORF) within said nucleotide sequence stored on a computer readable medium.

56. The method of claim 49, wherein said target sequence is a nucleotide sequence of between about 30 and about 300 nucleotide residues in length.

57. The method of claim 49, wherein said target sequence is identified as homologous to a sequence encoding an Emericella nidulans protein or fragment thereof within said one or more nucleotide sequences stored on a computer readable medium.

58. A method of detecting a nucleotide sequence using a computer, said method comprising:
a) providing a target nucleotide sequence;
b) comparing said target nucleotide sequence to one or more nucleotide sequences stored in a computer readable medium having recorded thereon at least 100 nucleotide sequences including at least one sequence selected from the group consisting of SEQ ID NO: 16207 through SEQ ID NO: 18349, SEQ ID NO: 18351 through SEQ ID NO: 18356, SEQ ID NO: 18358 through SEQ ID NO: 21010, and SEQ ID NO: 21012 through SEQ ID NO: 27905 and complements thereof, wherein said target sequence is compared to at least one sequence selected from the group consisting of SEQ ID NO: 16207 through SEQ ID NO: 18349, SEQ ID NO: 18351 through SEQ ID NO: 18356, SEQ ID NO: 18358 through SEQ ID NO: 21010, and SEQ ID NO: 21012 through SEQ ID NO: 27905; and
c) identifying said target sequence as homologous to said nucleotide sequence based on said comparison, wherein at least one of said comparing and identifying steps is carried out via the computer.

59. The method according to claim 58, wherein said target sequence is homologous to an open reading frame (ORF) within said nucleotide sequence.

60. The method of claim 58, wherein said target sequence is a nucleotide sequence of between about 30 and about 300 nucleotide residues in length.

61. The method of claim 58, wherein said target sequence is identified according to degree of homology to said nucleotide sequence stored in a computer readable medium.

62. A method of ranking a target nucleotide sequence by homology to a nucleotide sequence of E. nidulans using a computer-based system, said method comprising:
a) providing a target nucleotide sequence to a computer-based system having search means comprising a program to compare a target nucleotide sequence to nucleotide sequences stored on data storage means having recorded thereon at least 100 nucleotide sequences including at least one sequence selected from the group consisting of SEQ ID NO: 16207 through SEQ ID NO: 18349, SEQ ID NO: 18351 through SEQ ID NO: 18356, SEQ ID NO: 18358 through SEQ ID NO: 21010, and SEQ ID NO: 21012 through SEQ ID NO: 27905 and complements thereof;
b) using said search means to compare said target nucleotide sequence to said nucleotide sequences stored on data storage means, wherein said target nucleotide sequence is compared to at least one sequence selected from the group consisting of SEQ ID NO: 16207 through SEQ ID NO: 18349, SEQ ID NO: 18351 through SEQ ID NO: 18356, SEQ ID NO: 18358 through SEQ ID NO: 21010, and SEQ ID NO: 21012 through SEQ ID NO: 27905; and
c) ranking said target sequence based on percent homology to said nucleotide sequence of E. nidulans, wherein at least one of said comparing and ranking steps is carried out by the computer.

63. The method of claim 62, wherein said target sequence is a nucleotide sequence of between about 30 and about 300 nucleotide residues in length.

64. The method of claim 62, wherein said search means constructs an optimal alignment for each region of the target nucleotide sequence and a nucleotide sequence stored on data storage means.

65. A method for identifying a nucleic acid sequence using a computer, said method comprising:
a) providing a target nucleotide sequence;
b) comparing said target nucleotide sequence to one or more nucleotide sequences stored in a computer readable medium having recorded thereon at least 100 nucleotide sequences including at least one sequence selected from the group consisting of SEQ ID NO: 16207 through SEQ ID NO: 18349, SEQ ID NO: 18351 through SEQ ID NO: 18356, SEQ ID NO: 18358 through SEQ ID NO: 21010, and SEQ ID NO: 21012 through SEQ ID NO: 27905 and complements thereof, wherein said target nucleotide sequence is compared to at least one of said sequences selected from the group consisting of SEQ ID NO: 16207 through SEQ ID NO: 18349, SEQ ID NO: 18351 through SEQ ID NO: 18356, SEQ ID NO: 18358 through SEQ ID NO: 21010, and SEQ ID NO: 21012 through SEQ ID NO: 27905; and
c) identifying said target nucleotide sequence as having significant sequence identity to said one or more nucleotide sequences stored in a computer readable medium, wherein said sequences stored in said computer readable medium function to facilitate said identification of said target sequence as having significant sequence identity, wherein at least one of said comparing and identifying steps is carried out via the computer.

66. The method of claim 65, wherein said method identifies a nucleic acid sequence within the Emericella nidulans genome.

67. The method of claim 65, wherein said target sequence shares between 100% and 90% sequence identity with one or more of said sequences selected from the group consisting of SEQ ID NO: 16207 through SEQ ID NO: 18349, SEQ ID NO: 18351 through SEQ ID NO: 18356, SEQ ID NO: 18358 through SEQ ID NO: 21010, and SEQ ID NO: 21012 through SEQ ID NO: 27905.

68. The method of claim 67, wherein said target sequence shares between 100% and 95% sequence identity with one or more of said sequences selected from the group consisting of SEQ ID NO: 16207 through SEQ ID NO: 18349, SEQ ID NO: 18351 through SEQ ID NO: 18356, SEQ ID NO: 18358 through SEQ ID NO: 21010, and SEQ ID NO: 21012 through SEQ ID NO: 27905.

69. The method of claim 68, wherein said target sequence shares between 100% and 98% sequence identity with one or more of said sequences selected from the group consisting of SEQ ID NO: 16207 through SEQ ID NO: 18349, SEQ ID NO: 18351 through SEQ ID NO: 18356, SEQ ID NO: 18358 through SEQ ID NO: 21010, and SEQ ID NO: 21012 through SEQ ID NO: 27905.

70. The method of claim 69, wherein said target sequence shares between 100% and 98% sequence identity with one or more of said sequences selected from the group consisting of SEQ ID NO: 16207 through SEQ ID NO: 18349, SEQ ID NO: 18351 through SEQ ID NO: 18356, SEQ ID NO: 18358 through SEQ ID NO: 21010, and SEQ ID NO: 21012 through SEQ ID NO: 27905.

71. A method for identifying the function of a fungal nucleic acid sequence by determining homology to a nucleotide sequence in the Emericella nidulans genome using a computer-based system, said method comprising:
a) providing a target fungal nucleotide sequence to a computer-based system having a homology-based search program;
b) using said homology-based search program to compare said target fungal nucleotide sequence to one or more E. nidulans nucleotide sequences stored in said computer-based system having recorded thereon at least 100 nucleotide sequences including at least one sequence selected from the group consisting of SEQ ID NO: 16207 through SEQ ID NO: 18349, SEQ ID NO: 18351 through SEQ ID NO: 18356, SEQ ID NO: 18358 through SEQ ID NO: 21010, and SEQ ID NO: 21012 through SEQ ID NO: 27905 and descriptions identifying encoded proteins, wherein said target fungal nucleotide sequence is compared to at least one of said sequences selected from the group consisting of SEQ ID NO: 16207 through SEQ ID NO: 18349, SEQ ID NO: 18351 through SEQ ID NO: 18356, SEQ ID NO: 18358 through SEQ ID NO: 21010, and SEQ ID NO: 21012 through SEQ ID NO: 27905 and provide a rank based on homology; and
c) identifying the function of said target nucleotide sequence based on homology to a nucleotide sequence in the E. nidulans genome based on said rank.

72. A method of identifying a nucleotide sequence using a computer, said method comprising comparing a target sequence to one or more sequences stored in computer readable medium having recorded thereon at least 100 nucleotide sequences including at least one sequence selected from the group consisting of SEQ ID NO: 16207 through SEQ ID NO: 18349, SEQ ID NO: 18351 through SEQ ID NO: 18356, SEQ ID NO: 18358 through SEQ ID NO: 21010, and SEQ ID NO: 21012 through SEQ ID NO: 27905 and complements thereof, and identifying said target sequence as having significant sequence identity to at least one sequence selected from the group consisting of SEQ ID NO: 16207 through SEQ ID NO: 18349, SEQ ID NO: 18351 through SEQ ID NO: 18356, SEQ ID NO: 18358 through SEQ ID NO: 21010, and SEQ ID NO: 21012 through SEQ ID NO: 27905, wherein both of said comparing and identifying steps are carried out via the computer.
